(19) United States
(12) Patent Application Publication
(10) Pub. No.: US 2014/0221802 A1
(43) Pub. Date: Aug. 7, 2014

(54) ONE TOUCH LASER LANCING TYPE BLOOD GLUCOSE MEASUREMENT DEVICE, STRIP AND THE METHOD USING IT

(71) Applicant: Isotech Co., Ltd., Anyang-si (KR)

(72) Inventor: Kee Jung CHOI, Anyang-si (KR)

(73) Assignee: Isotech Co., Ltd., Anyang-si (KR)

(21) Appl. No.: 14/244,692

(22) Filed: Apr. 3, 2014

Related U.S. Application Data
(63) Continuation-in-part of application No. 13/061,368, filed on Feb. 28, 2011, now abandoned.

(30) Foreign Application Priority Data
Aug. 27, 2008 (KR) 10-2008-0083705

(51) Int. Cl. A61B 5/151; A61B 5/157; A61B 5/15; A61B 5/145

(52) U.S. Cl. CPC A61B 5/15136; A61B 5/14532; A61B 5/157; A61B 5/150358
USPC 600/365

(57) ABSTRACT

A one touch laser lancing type blood glucose measurement device, a strip, and the method using it. In particular, the invention relates to a one touch laser lancing type blood glucose measurement device, a strip, and the method using it, which performs blood gathering and blood glucose measurement at the same time, using laser beams generated in a laser module. The laser beams are irradiated into a finger passing a hole of a strip with a focusing lens converging the laser beams, a protect window, and the hole to gather blood, then the blood is absorbed into the strip for measuring blood glucose.

### CROSS-REFERENCE TO RELATED APPLICATION

This is a continuation-in-part application of U.S. patent application Ser. No. 13/061,368 filed on Feb. 28, 2011, which claims the benefit of Korean Patent Application No. 10-2008-0083705, filed on Aug. 27, 2008, the entire disclosures of which are incorporated herein by reference.

### FIELD OF THE INVENTION

The invention relates to a one touch laser lancing type blood glucose measurement device, a strip, and the method using it. In particular, the invention relates to a one touch laser lancing type blood glucose measurement device, a strip, and the method using it, which performs blood gathering and blood glucose measurement at the same time, using laser beams generated in a laser module. The laser beams are irradiated to a finger passing a hole of a strip with a focusing lens converging the laser beams, a protect window, and the hole to gather blood, then the blood is absorbed into the strip for measuring blood glucose.

### BACKGROUND OF THE INVENTION

Recently, the International Diabetes Federation (IDF) warned that diabetics number over about 360 million around the globe, and the number of diabetics increases about 8.6% every year, therefore solutions by the government are required.

For diagnosing diabetes, the density of glucoses in blood plasma of blood components is measured, and management of blood sugar is very important.

Generally, blood-gathering and blood glucose measurement devices prick the skin surface by using a lancing device with a lancet having a metal needle shape. Thus, diabetics feel pain and have scars, and a superfluous amount of blood is gathered and dropped onto a strip end. Then, the strip is inserted into a blood glucose measurement device separately formed, to measure blood sugar level.

As described above, for measuring blood sugar level, the lancet, the lancing device, the blood glucose measurement device, the strip, and alcohol wipes are needed.

However, due to excessive TV watching and irregular diet habits, the number of children suffering from diabetes increases. The lancet having the needle causes pain and fear in children and adults.

In addition, a diabetic relying on insulin injections who measures the blood sugar level 2 to 4 times every day must have a blood glucose measurement device, but it is onerous to carry the various components of the blood glucose measurement device.

To solve these problems, a blood glucose measurement device apparatus comprising a lancet, a lancing device and a blood-gathering device using laser beams has been developed. Blood is gathered using laser beams in one end of the apparatus and is dropped onto the strip, then the strip is inserted into a strip connector to measure blood sugar level in another end of the apparatus.

However, even though the apparatus has both a blood-gathering function and a blood sugar level measuring function, blood is gathered by inserting a disposal cap into one end of the apparatus, while the strip is inserted into another end of the apparatus for measuring blood sugar level. Thus, using the apparatus is not easy. To avoid pollution of the blood glucose measurement device, a disposal cap covered with a film is used, but has to be carried.

Since the apparatus has a blood glucose measurement device and a blood-gathering device on opposite ends, the volume of the apparatus increases. When the diabetic relying on insulin injections is a child, controlling the apparatus is difficult due to the large volume.

### BRIEF SUMMARY OF THE INVENTION

An embodiment of the invention provides a one touch laser lancing type blood glucose measurement device, a strip, and the method using it performing a blood-gathering process, a process of sucking blood with the strip, and a process of measuring blood glucose level by a one button operation at the same time in a blood glucose measurement device and the strip.

Another embodiment of the invention provides a one touch laser lancing type blood glucose measurement device, a strip, and the method using it with decreased volume by combining a blood-gathering device and a blood glucose measurement device.

Another embodiment of the invention provides a strip forming a film on an upper end of a strip hole in order to avoid pollution of the device.

A one touch laser lancing type blood glucose measurement device according to the invention comprises a laser module generating laser beams and irradiating the laser beams in one direction; a focusing lens converging the laser beams generated by the laser module and disposed in a laser irradiation direction; a protect window disposed in a laser direction progressing through the focusing lens and protecting the focusing lens from foreign bodies; a controlling unit disposed on a position close to a lateral side of the laser module and controlling a blood measure and the laser beams; a strip connector connected to the controlling unit and formed in a position on which a strip hole and the protect window are coincided with each other, and inserting a strip; a body unit containing the laser module, the focusing lens, the protect window, the controlling unit, and the strip connector; an interlock disposed on the outside of the body unit, positioned on the upper or lower side of the strip inserted into the strip connector, forming a hole, and changing the laser module by pressing with a finger into a ready state; and a button unit formed on a lateral side different form or an opposite side to the interlock holder and operating the laser module.

The invention further comprises a finger interlock formed on the outside of the body unit, preventing the laser beams irradiated from the laser module from being exposed to the exterior, and having a shape, which a finger is inserted into and a first sensor making a user sense the finger interlock's existence.

The invention further comprises a second sensing unit sensing whether the state of the laser module is changed into the ready state.

In a strip used in a blood glucose measurement device according to the invention, the strip comprises a reagent unit; an electrode unit inserted into strip connector to transfer blood information to the blood glucose measurement device; a strip hole passing laser beams irradiated from the laser module to irradiate the laser beams; and a polypropylene film included on the upper of the strip hole to prevent the blood glucose measurement device from becoming polluted.

The reagent unit measures a glucose from among the group consisting of a ferment, anti-insulin, and HbAlc.

A method for measuring blood glucose using a one touch laser lancing type blood glucose measurement device according to the invention comprises a first process of inserting a strip into a strip connector; a second process of inserting a finger into an upper of an interlock holder and into the inner of the finger lock holder; a third process of changing into a ready state by pressing the interlock holder; a fourth process of operating a laser module using a button module to generate laser beams, after the change into the ready state; a fifth process of converging the generated laser beams into a focusing lens, passing a protect window, a strip hole, and an interlock holder, and then irradiating the laser beams into the inserted finger; a sixth process of causing blood oozed from the finger due to the irradiating laser beams to be absorbed into the strip; a seventh process of transmitting data regarding the absorbed blood to a controlling unit; and an eighth process of outputting analyzed data regarding the absorbed blood from the controlling unit to a displaying unit.

A one touch laser lancing type blood glucose measurement device, a strip, and the method using it according to the invention performs a blood-gathering process, a process of sucking blood with the strip, and a process of measuring blood glucose level by a one button operation at the same time in the blood glucose measurement device and the strip.

The invention is a mixed construction having a blood-gathering device and a blood glucose measurement device installed on one side, thereby decreasing the volume of the device. Thus, the one touch laser lancing type blood glucose measurement device is easily portable.

The invention blocks dust etc. absorbed on the focusing lens by irradiating high temperature laser beams generated in blood-gathering by laser beams and exactly measuring the blood sugar level using a small amount of blood.

### BRIEF DESCRIPTION OF THE DRAWINGS

The disclosure may best be understood by reference to the following description taken in conjunction with the following figures:

FIGS. 1A to 1E are diagrams representing a one touch laser lancing type blood glucose measurement device according to the invention;

FIG. 2 shows a diagram representing a laser module included in a blood-gathering using a one touch laser lancing type blood glucose measurement device according to the invention;

FIG. 3A shows a diagram representing blood-gathering using a one touch laser lancing type blood glucose measurement device according to the invention;

FIG. 3B shows that blood gathered using a one touch laser lancing type blood glucose measurement device according to the invention is absorbed into a strip;

FIGS. 4A and 4B are diagrams showing a strip used in a one touch laser lancing type blood glucose measurement device according to the invention; and

FIG. 5 is a flow chart of a blood glucose measurement method using a one touch laser lancing type blood glucose measurement according to the invention.

### DETAILED DESCRIPTION OF THE INVENTION

Hereinafter, embodiments of the invention will be described in detail referring to the accompanying drawings.

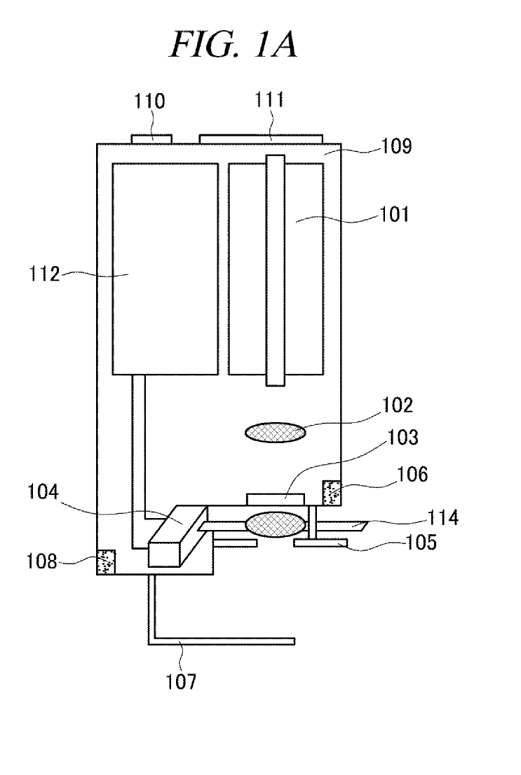

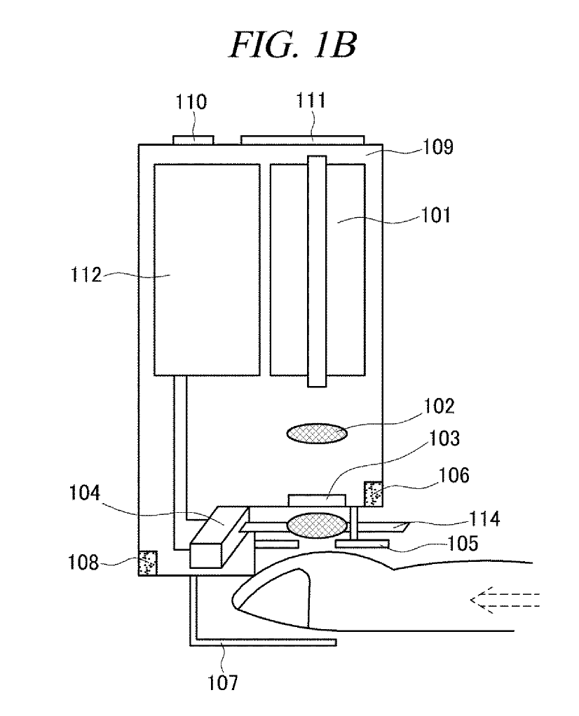

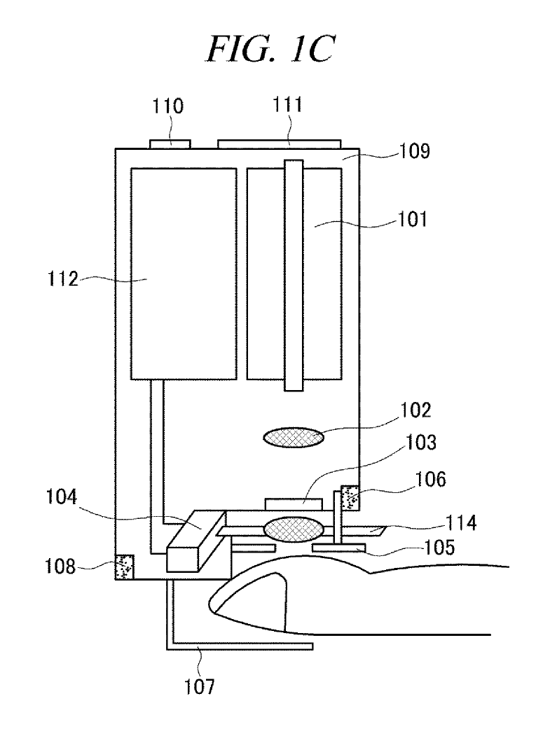

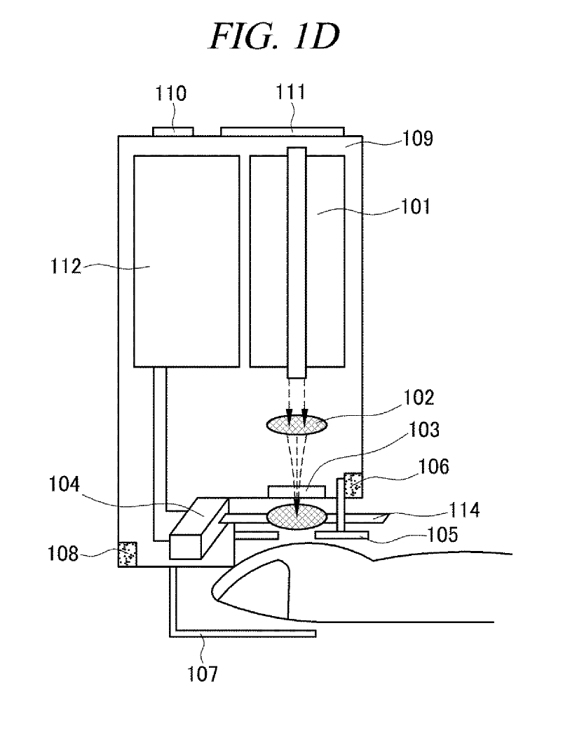

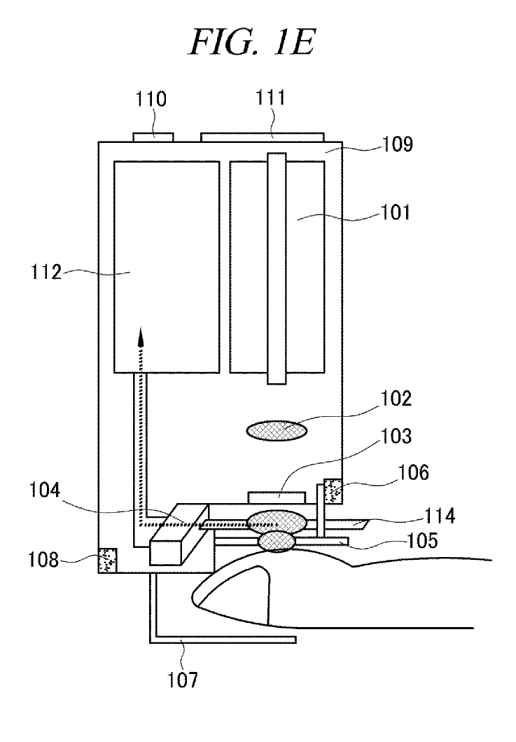

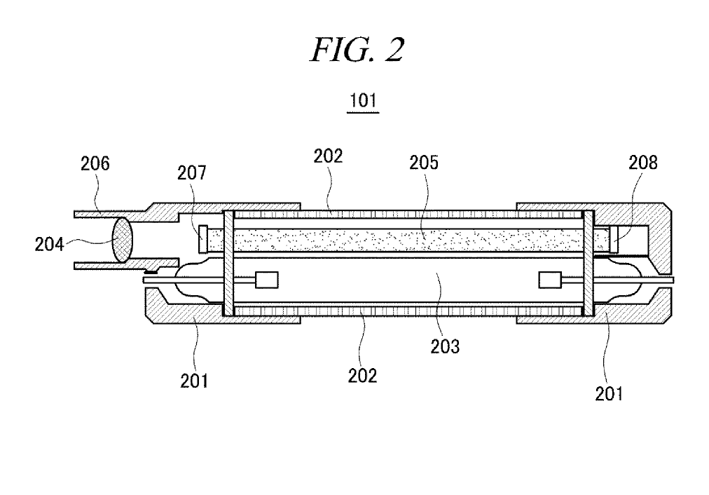

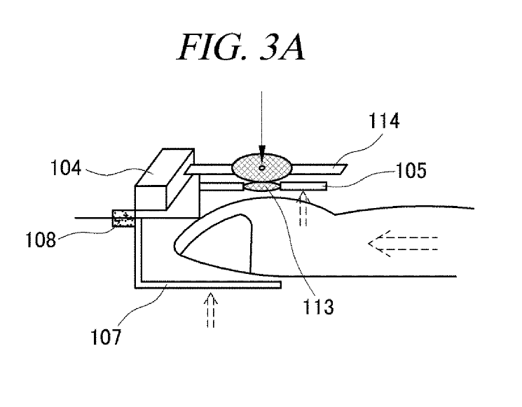

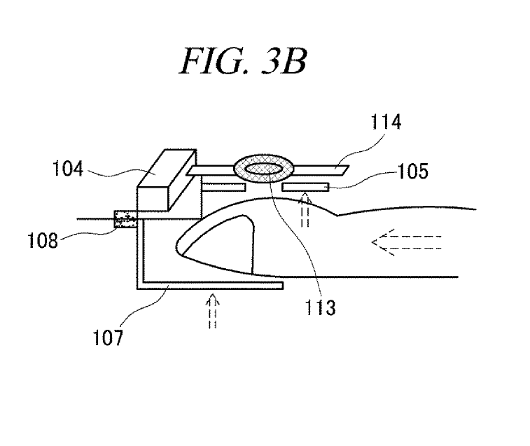

FIG. 1A to 1E are diagrams representing a one touch laser lancing type blood glucose measurement device according to the invention, FIG. 2 shows a diagram representing a laser module included in a blood-gathering using a one touch laser lancing type blood glucose measurement device according to the invention, FIG. 3A shows a diagram representing blood-gathering using a one touch laser lancing type blood glucose measurement device according to the invention, and FIG. 3B shows that blood gathered using a one touch laser blood lancing type glucose measurement device according to the invention is absorbed into a strip.

As shown in FIGS. 1A to 3, a one touch laser lancing type blood glucose measurement device comprises a laser module 101, a focusing lens 102, a protect window 103, a controlling unit 112, a strip connector 104, a body unit 10, an interlock holder 105, a second sensing unit 106, a button unit 110, a finger interlock 107, a first sensing unit 108, and a displaying unit 111.

The laser module 101 generates laser beams and irradiates the laser beams in a single direction to help a user gather blood in a user's finger. The laser beams generated in the laser module 101 are an Er:YAG laser technique and may gather the blood 113 in skin without a scar. The laser beams also have a wavelength of 2.94 um that has a strong absorbing power with respect to water and leaves no poisons in the body.

In some embodiments, as depicted in FIG. 2, the laser module 101 includes a laser module holder 201, a reflector 202, a xenon flash lamp 203, a focusing lens 204, an Er:YAG crystal rod 205, and a lens holder 206. The laser module holder 201 accommodates and holds the reflector 202, xenon flash lamp 203, focusing lens 204, Er:YAG crystal rod 205 and lens holder 206. The reflector 202 reflects light energy (i.e., laser) that is generated from the xenon flash lamp 203 by internal reflection. Thus, the reflector 202 increases transmission efficiency of the light energy (i.e., laser) from the xenon flash lamp 203 to the Er:YAG crystal rod 205. The xenon flash lamp 203 generates and/or emits the light energy as a pumping source of the light energy. Specifically, a photo condenser (not illustrated) that may be charged with high voltage is connected to the xenon flash lamp 203. The photo condenser applies the charged high voltage to the xenon flash lamp 203 via a trigger coil (not illustrated). Then, insulation of xenon gas in the xenon flash lamp 203 is broken by the applied high voltage, and the light energy is generated and emitted upon the insulation of xenon gas being broken. The Er:YAG crystal rod 205 receives and/or absorbs the light energy emitted from the xenon flash lamp 203. Further, an 85% reflection mirror 207 having reflectivity of 85 percent and a 100% total reflection mirror 208 that has reflectivity of 100 percent (i.e., total reflection) and faces to the 85% reflection mirror 207 are attached to the Er:YAG crystal rod 205. The absorbed light energy resonates in the Er:YAG crystal rod 205 by the 85% reflection mirror 207 and 100% total reflection mirror 208. Then, the laser beams having a wavelength of 2940 nm is emitted based on the resonance of the light energy. The focusing lens 204 concentrates the laser beams emitted from the Er:YAG crystal rod 205. The lens holder 206 holds the focusing lens 204.

In the invention, the Er:YAG laser technique is described as an embodiment. However, the invention is not limited to the Er:YAG laser technique and a laser technique having the characteristics equal to the Er:YAG laser technique and not harmful to the body may be used.

The focusing lens 102 converges the laser beams into a limited pricked skin part. In more detail, the focusing lens 102 is an element that converges the laser beams progressing in a predetermined direction to blood-gathering object skin, that is, a finger with an accurate focus. Thereby, the focusing lens 12 may prick to make a hole having a diameter of 0.1 mm to 0.5 mm and a depth of 0.2 mm to 4 mm. However, to avoid painful sores by stimulation of nerve cells into the true skin, it is preferable that the focusing lens 12 may prick to make a hole having a depth of 0.5 mm or less and diameter of 0.3 mm or less.

The focusing lens 102 may also use a convex lens or an aspherical lens accurately to focus the laser beams to the pricked skin part.

The protect window 103 protects the focusing lens 102 from foreign bodies in the direction in which the laser beams from the focusing lens 102 progress. The protect window 103 is spaced apart from the focusing lens 102 by a predetermined distance in the laser progressing direction.

The controlling unit 112 controls a blood sugar level measurement of blood input into a position close to a lateral side of the laser module 101 and the laser beams.

Further, the controlling unit 112 controls operations of the laser module 101, the strip connector 104, the first sensing unit 108, the second sensing unit 106, and the displaying unit 111 so as to measure the blood sugar level in blood and to provide the blood sugar level to a user. The controlling unit 112 includes a power supply to supply power; and a control printed circuit board (PCB) to control the laser module 101, the strip connector 104, the first sensing unit 108, the second sensing unit 106, and the displaying unit 111. The controlling unit 112 controls the laser module 101 to generate and irradiate the laser beams when the laser module 101 is in a ready state for irradiating the laser beams. For example, the controlling unit 112 controls the laser module 101 to charge a photo condenser with a predefined voltage and to generate the laser beams by the voltage charged in the photo condenser. Further, the controlling unit 112 controls the strip connector 104 to transfer, to the controlling unit 112, blood data regarding the blood absorbed into a strip 114 and receives the transferred blood data. Further, the controlling unit 112 calculates and/or measures the blood sugar level, based on the received blood data. Further, the controlling unit 112 controls the first sensing unit 108 to determine and/or sense whether the strip 114 is inserted into the strip connector 104. Further, the controlling unit 112 controls the second sensing unit 106 to determine and/or sense whether a state of the laser module 101 is changed into the ready state when the interlock holder 105 is pressed by a finger. Further, the controlling unit 112 controls the displaying unit 111 to display the measured blood sugar level.

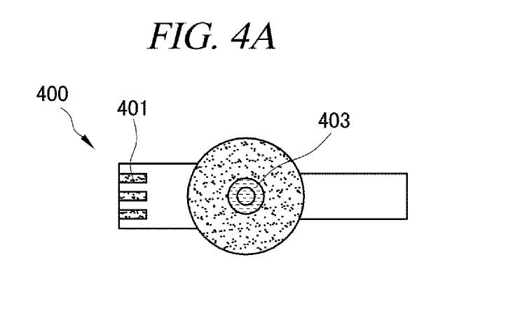

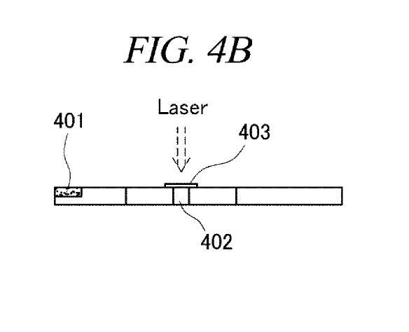

The strip connector 104 connects to the controlling unit 112, but the strip connector 104 is formed in a position on which a strip hole and the protect window 103 are coincided with each other and inserts a strip 114. The used strips 114 and 400 will be described in detail later, referring to FIG. 4. The strip connector 104 obtains data of the blood 113 in the strip 114 to transfer the data to the controlling unit 12.

The body unit 109 contains the laser module 101, the focusing lens 102, the protect window 103, the controlling unit 112, and the strip connector 104. At this time, the strip connector 104 is contained into the body unit 109, but it is evident that a gate for inserting the strip 114 is exposed to the outside from the body unit 109.

The interlock holder 105 is disposed on the outside of the body unit 19, and positioned on the upper or lower side of the strip 114 inserted into the strip connector 104. The interlock holder 105 forms a hole. The hole disposes the strip hole 402 formed in the strip, the protect window 103, and the focusing lens 102 on a straight line, and then is pressed by a finger. Thereby, a state of the laser module 101 is changed into a ready state. At this time, the strip hole 402 is shown in FIG. 4.

When the state of the interlock holder is changed into the ready state, laser beams may be generated using a button. However, when the state of the interlock holder is not changed into the ready state, laser beams may not generated regardless of a state of the button, to protect the user. The Er:YAG laser technique is not harmful to most parts of a human body. However, when the laser beams generated by the Er:YAG laser technique irradiates human eyes, the eyes may be irreversibly damaged. Therefore, the embodiments described herein may prevent accidents by changing the state of the interlock holder.

The second sensing unit 106 determines and/or senses whether the laser module 101 is in the ready state. When a finger is inserted into the finger interlock 107, as depicted in FIG. 1B, the interlock holder 105 is pressed by the inserted finger. Thus, the interlock holder 105 moves up toward the second sensing unit 106 as depicted in FIG. 1C and touches a micro switch (not illustrated) included in the second sensing unit 106. The second sensing unit 106 determines that the laser module 101 is in the ready state, if the interlock holder 105 touches the micro switch. The second sensing unit 106 is configured to senses when the laser module 101 is in the ready state by using the micro switch. Other sensing devices to detect an object, such as a photo sensor, a push button sensor, infrared sensor, etc. may be included in the second sensing unit 106 to determine the state of the laser module 101.

The finger interlock 107, formed on the outside of the body unit 109, prevents laser beams generated in the laser module 101 from being exposed to the exterior, and is shaped to receive a finger inserted thereto. Since the finger interlock 107 may be inserted inward of body unit 109, when the blood glucose measurement device is not used, it may be easily stowed and/or carried.

The first sensing unit 108 senses whether the finger interlock 107 is opened. When the finger interlock 107 is damaged, laser beams may be exposed to the exterior. Thus, when the finger interlock 107 is damaged, the first sensing unit 108 operates to stop the operations of the blood glucose measurement device, to function as a safety device. Further, the first sensing unit 108 receives, from the strip connector 104, an electric signal when the strip 114 is inserted into the strip connector 104. Further, the first sensing unit 108 determines that the strip 114 is inserted into the strip connector 104, upon receiving the electric signal.

The button unit 110 includes one or more pushable buttons formed on a different position from the interlock holder 105 to operate the laser module 101. The button unit 110 is able to operate only when the interlock holder 105 is in the ready state, for the user's safety. The laser beam is irradiated to the finger from the laser module 101, when one or more of the buttons corresponding to button unit 110 is pressed. Thereby blood 120 may discharge from the finger as depicted in FIG. 1D.

The displaying unit 111 outputs data measured by the operating of the laser module 101 by the button unit 10 and by the transferring the blood 113 obtained by the strip 114 to the controlling unit 112 through the strip connector 104 as depicted in FIG. 1E.

As described herein, since the blood-gathering device and the blood glucose measurement device are on one side and complexly formed, there is an advantage that the blood-gathering and the blood sugar level measurement are performed by a one button operation.

FIGS. 4A and 4B are diagrams showing a strip used in a one touch laser lancing type blood glucose measurement device according to the invention.

As shown in FIGS. 4A and 4B, a strip 400 inserted into the strip connector used in the invention comprises an electrode unit 401, a strip hole 402, a reagent unit (not shown), and a PP film 403.

The electrode unit 401 is inserted into the strip connector 104 to transfer blood information to the controlling unit of the blood glucose measurement device. The electrode unit 401 includes one or more electrodes or chips to be inserted into the strip connector 104.

The strip hole 402 passes laser beams generated by the laser module to irradiate the laser beams and transfers the blood to the reagent unit. At this time, preferably, a diameter of the strip hole 4 is 3 mm, but the invention is not limited to that.

It is preferable that the reagent unit (not shown) may measure a glucose from among the group consisting of a ferment, anti-insulin, and HbAlc. In addition, the invention using the strip may measure at least one of ketone and cholesterol.

The PP (poly propylene) film 403 is included on the upper of the strip hole to prevent the blood glucose measurement device from becoming polluted. It is preferable that the PP film 403 has a thickness of 0.05 mm to 0.15 mm, and it is more preferable that the PP film 403 has a thickness of 0.1 mm.

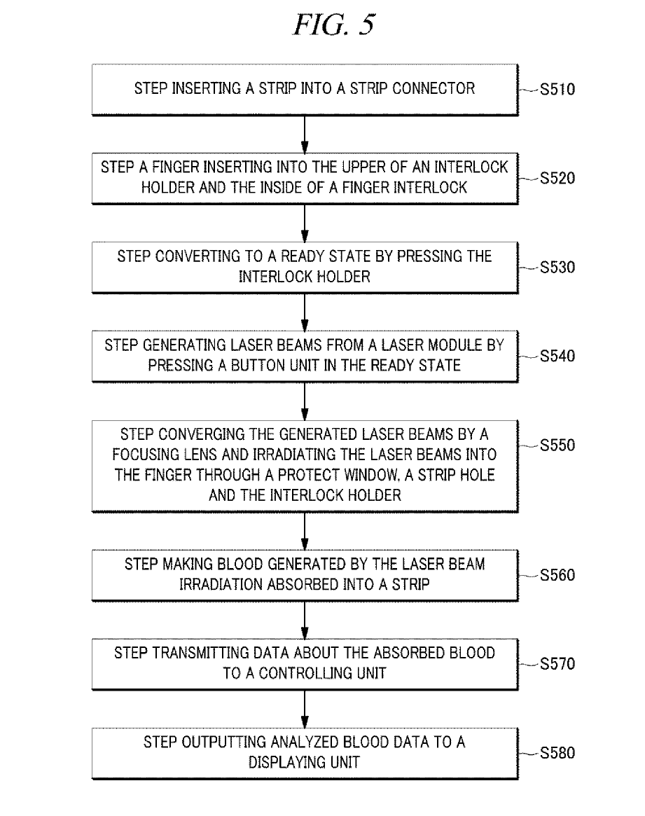

FIG. 5 is a flow chart of a blood glucose measurement method using a one touch laser lancing type blood glucose measurement according to the invention.

As shown in FIG. 5, the blood glucose measurement method using a one touch laser lancing type blood glucose measurement according to the invention will be described below.

First, the strip is inserted into the strip connector (S510). At this time, the strip hole is coincided with the focus lens and the protect window by the strip inserted into the strip

Sequentially, a finger is inserted into the upper of the interlock holder and the inner of the finger interlock (S520).

Sequentially, the interlock holder is pressed to be changed and converted into a ready state (S530). At this time, in being charged into the ready state, the displaying unit displays the time point in which it is possible to press the button unit.

Sequentially, the ready state is displayed in the displaying unit and a user presses one or more of the buttons of the button unit to operate the laser module. When the button unit is pressed in the ready state by the user, laser beams are generated by the laser module (S540).

Sequentially, the generated laser beams are converged onto the focusing lens and irradiated through the protect window, the strip hole and the interlock holder into the finger (S550). The laser beams irradiated into the finger cause blood to ooze from the finger.

The principle of the blood oozing caused by the laser beams is as below.

When the laser module generates laser beams of a high temperature to irradiate into the finger, the skin of the finger is burned by the laser beams to stimulate capillaries of the inner periphery and burst them. Thereby blood oozes from the finger.

It is preferable that the temperature of the laser beams is in the range from 1000.degree. C. to 1300.degree. C. Since the laser beams have a high temperature, it is possible to perform the blood-gathering without a disinfection operation.

Sequentially, the blood that is gathered by the irradiation of the laser beams is absorbed into the strip (S560).

Sequentially, data regarding the blood absorbed into the strip is transferred to the controlling unit. At this time, a glucose from among the group consisting of a ferment, anti-insulin, and HbAlc may be measured (S570).

Finally, the controlling unit outputs analyzed data regarding the blood to the displaying unit (S580).

While this invention has been described in connection with what is considered to be practical exemplary embodiments, it is to be understood that the invention is not limited to the disclosed embodiments, but, on the contrary, is intended to cover various modifications and equivalent arrangements included within the spirit and scope of the appended claims.

The above description of the present invention is provided for the purpose of illustration, and it would be understood by those skilled in the art that various changes and modifications may be made without changing technical conception and essential features of the present invention. Thus, it is clear that the above-described embodiments are illustrative in all aspects and do not limit the present invention.

The scope of the present invention is defined by the following claims rather than by the detailed description of the embodiment. It shall be understood that all modifications and embodiments conceived from the meaning and scope of the claims and their equivalents are included in the scope of the present invention.

### DESCRIPTION OF REFERENCE CHARACTERS

101: laser module
102: focusing lens
103: protect window
104: strip connector
105: interlock holder
106: second sensing unit 106
107: finger interlock
108: first sensing unit
109: body unit
110: button unit
111: displaying unit
112: controlling unit
113: blood
114: strip
201: laser module holder
202: reflector
203: xenon flash lamp
204: focusing lens
205: Er:YAG crystal rod
206: lens holder
207: 85% reflection mirror
208: 100% total reflection mirror
400: strip
401: electrode unit
402: strip hole
403: polypropylene film

What is claimed is:

1. A one touch laser lancing type blood glucose measurement device comprising:
a laser module configured to generate laser beams and irradiate the laser beams;
a focusing lens that is configured to converge the laser beams generated from the laser module and is disposed in a laser irradiation direction;
a protecting window disposed between a strip to be inserted and the focusing lens and configured to protect the focusing lens from foreign bodies;
a controlling unit disposed at a position close to a lateral side of the laser module and configured to control a blood measure and the laser module;
a strip connector connected to the controlling unit and placed at a position for inserting the strip such that a strip hole of the strip and the protecting window are aligned with each other vertically;
a body unit configured to contain the laser module, the focusing lens, the protecting window, the controlling unit, and the strip connector;
an interlock holder disposed on the outside of the body unit, positioned on the upper or lower side of the strip, configured to receive a fingerprint side of a finger and change a state of the laser module into a ready state when the interlock holder is pressed by the fingerprint side of the received finger; and
a button unit disposed on the body unit and configured to operate the laser module.

2. The device of claim 1, further comprising:
a finger interlock formed on the outside of the body unit, configured to prevent the laser beams irradiated from the laser module from being exposed to the exterior of the finger interlock, and configured to have a shape adapted to receive a fingernail side of the finger; and
a first sensing unit configured to sense whether the finger interlock is opened.

3. The device of claim 1, further comprising:
a second sensing unit configured to sense whether the state of the laser module is changed into the ready state.

4. A strip for use in a blood glucose measurement device, the strip comprising:
an electrode unit provided at an end of the strip and configured to be inserted into a strip connector to transfer blood information to the blood glucose measurement device;
a strip hole configured to pass laser beams from a laser module therethrough; and
a polypropylene film included on the upper of the strip hole to prevent the blood glucose measurement device from becoming polluted.

5. A blood glucose measurement method using the laser lancing type blood glucose measurement device of claim 2, the method comprising:
a first process of inserting the strip into the strip connector;
a second process of sensing whether the finger interlock is opened and inserting a finger into a gap between the interlock holder and the finger interlock if the finger interlock is opened;
a third process of changing a state of the laser module into the ready state by pressing the interlock holder with the inserted finger;
a fourth process of operating the laser module to generate laser beams;
a fifth process of converging the generated laser beams through the focusing lens through the protecting window, the strip hole, and the interlock holder, so as to irradiate the laser beams into the inserted finger to cause blood to ooze from the finger;
a sixth process of absorbing the blood oozed from the finger due to the irradiating laser beams into the strip;
a seventh process of transmitting data regarding the absorbed blood to the controlling unit; and
an eighth process of outputting analyzed data regarding the absorbed blood from the controlling unit.
